(19) United States
(12) Patent Application Publication
(10) Pub. No.: US 2019/0275352 A1
(43) Pub. Date: Sep. 12, 2019

(54) TREATMENT PLANNING BASED ON MULTIPLE MODALITIES

(71) Applicant: THE ROYAL INSTITUTION FOR THE ADVANCEMENT OF LEARNING/MCGILL UNIVERSITY, Montreal (CA)

(72) Inventors: Francois DEBLOIS, Montreal-Est (CA); Marc-Andre RENAUD, Montreal (CA); Jan SEUNTJENS, Westmount (CA)

(21) Appl. No.: 16/361,435

(22) Filed: Mar. 22, 2019

Related U.S. Application Data
(63) Continuation of application No. PCT/CA2017/051127, filed on Sep. 25, 2017.
(60) Provisional application No. 62/398,785, filed on Sep. 23, 2016.

(51) Int. Cl. A61N 5/10; G16H 50/20

(52) U.S. Cl. CPC A61N 5/1038; A61N 2005/1034; G16H 50/20

(57) ABSTRACT

There is described herein methods and systems for generating a treatment plan for delivery of a radiation dose to a subject based on a plurality of radiation modalities, each radiation modality having a delivery element and an associated weight associated thereto. The treatment plan is constructed iteratively by considering the different radiation modalities and different delivery elements and selecting those that meet one or more goals regarding a target dose distribution.

### CROSS-REFERENCE TO RELATED APPLICATIONS

The present application is a continuation of International Patent Application No. PCT/CA2017/051127 filed on Sep. 25, 2017, which claims the benefit of U.S. Provisional Patent Application No. 62/398,785 filed on Sep. 23, 2016, the contents of which are hereby incorporated in their entirety by reference.

### TECHNICAL FIELD

The present disclosure relates generally to treatment plans for various medical conditions, and more particularly, to treatment plans for treatments that may be delivered via multiple modalities.

### BACKGROUND OF THE ART

Radiotherapy is used for the treatment of various medical conditions. For example, it can be used for the ablation or local control of cancerous lesions. Various modalities for the delivery of radiation may be used, alone or in combination. Automatic treatment planning is generally used to optimize the delivery of single modality treatments. Multiple modalities, however, are generally delivered non-optimally, in part due to various issues that arise when combining multiple modalities in a treatment planning process.

### SUMMARY

There is described herein methods and systems for generating a treatment plan for delivery of a radiation dose to a subject based on a plurality of radiation modalities, each radiation modality having a delivery element and an associated weight associated thereto. The treatment plan is constructed iteratively by considering the different radiation modalities and different delivery elements and selecting those that meet one or more goals regarding a target dose distribution.

In accordance with one broad aspect, there is provided a method for generating a treatment plan for delivery of a radiation dose to a subject. The method comprises obtaining at least one medical image of the subject; defining at least one goal regarding a target dose distribution to at least a portion of the at least one image; iteratively constructing the treatment plan by selecting at least one radiation modality from a plurality of radiation modalities, the at least one radiation modality having at least one delivery element from a plurality of delivery elements and at least one associated weight, until a condition associated with the at least one goal is met; and generating the treatment plan based on the at least one delivery element and at least one associated weight, for delivery of the radiation dose by the at least one radiation modality.

In some embodiments, iteratively constructing the treatment plan comprises combining at least two modalities from the plurality of modalities to satisfy the at least one goal.

In some embodiments, iteratively constructing the treatment plan comprises (a) determining a highest potential radiation modality from the plurality of radiation modalities, the highest potential modality having a greatest likelihood of reaching the at least one goal and having at least one delivery element from the plurality of delivery elements associated therewith; (b) adjusting at least one weight associated with the at least one delivery element to move towards the at least one goal; (c) determining an actual dose distribution on the at least one image using the at least one weight and at least one delivery element; and (d) adding, removing, or changing a radiation modality and repeating (b) and (c) until the condition associated with the at least one goal is met by the actual dose distribution.

In accordance with another broad aspect, there is provided a non-transitory computer-readable medium having program instructions stored thereon that are executable by a processor for performing the method generating a treatment plan for delivery of a radiation dose to a subject.

In accordance with yet another broad aspect, there is provided a system comprising at least one radiation modality and a computing system operatively connected to the at least one radiation modality. The computing system is configured to provide control signals to the computing system to deliver a radiation dose to a subject in accordance with the treatment plan generated using the method of generating a treatment plan for delivery of a radiation dose to a subject.

In some embodiments, the computing system is configured to send the control signals to a record and verify system.

In some embodiments, the system is configured to provide the control signals by direct upload of instructions to the at least one radiation modality.

### BRIEF DESCRIPTION OF THE DRAWINGS

Further features and advantages of the present invention will become apparent from the following detailed description, taken in combination with the appended drawings, in which:

FIG. 1 is a schematic illustration of a system arranged in accordance with at least some embodiments described herein;

FIG. 2A is a flowchart of an example method for treating a subject arranged in accordance with at least some embodiments of the present disclosure;

FIG. 2B is a flowchart of an example method for iteratively constructing a treatment plan;

FIG. 3 is a block diagram illustrating an example computing device that is arranged for providing a multi-modality treatment with the present disclosure; and

FIG. 4 is a block diagram illustrating an example computer program product that is arranged to store instructions for providing a multi-modality treatment in accordance with the present disclosure;

It will be noted that throughout the appended drawings, like features are identified by like reference numerals.

### DETAILED DESCRIPTION

This disclosure is drawn, inter alia, to methods, systems, products, devices, and/or apparatus generally related to the generation of treatment plans where two or more modalities may be considered for delivering the treatment plan. In some embodiments, the treatment plans are for the purposes of delivering radiation treatments. In some embodiments, the treatment plans are for the purposes of delivery multi-modality radiation treatments. However, it is to be understood that the treatment plans may be for delivering virtually any treatment where at least two treatment methods are available.

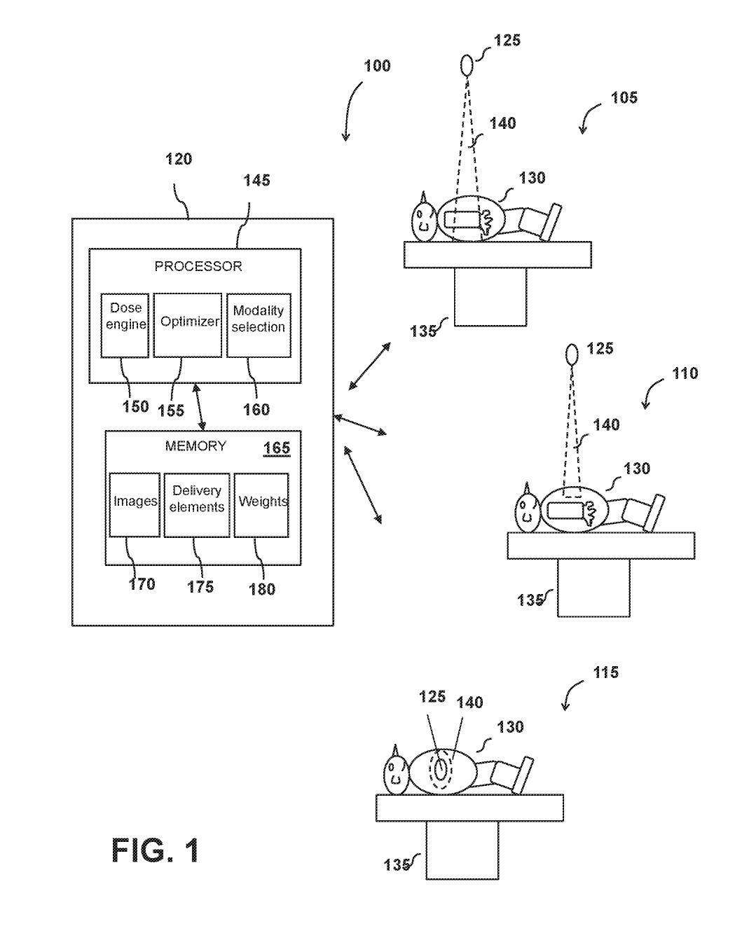

FIG. 1 is a schematic illustration of a system 100 arranged in accordance with at least some embodiments described herein. FIG. 1 shows multiple radiation modalities 105, 110 and 115 coupled with a computing system 120. The radiation modalities each have a radiation source 125 placed relative to a patient 130, who may be placed on a patient support apparatus 135. The radiation source 125 is generally used to treat a disease or condition of the patient, and configured to irradiate the suspected malignant anatomy with a radiation beam 140. The computing system 120 may include at least a processor 145, which may include a dose engine 150, an optimizer 155, and a modality selection unit 160. It may also include a memory 165, which may include images 170, a set of simulated delivery elements 175, and a set of weights 180. The various components described in FIG. 1 are merely examples, and other variations, including eliminating components, combining components, and substituting components are all contemplated.

The two or more radiation modalities 105, 110 and 115 differ in at least one of the following aspects: radiation (or particle) type, energy, and delivery mechanism. Some examples of radiation type are x-ray photons produced by medical linear accelerators or x-ray tubes, gamma ray photons produced by radionuclides such as Cobalt-60 or Iridium-192, electrons produced by medical particle accelerators, and protons, or carbon ions produced by synchrotrons or cyclotrons. Some examples of energies range from 6 MeV to 25 MeV and in some cases up to 200 MeV or more, or a spectrum of energies in that range (sometimes denoted as 6 MV to 25 MV or 50 MV to denote a spectrum rather than a monoenergetic energy). Some examples of delivery mechanisms are external beam radiotherapy, where the radiation source 125 is outside the patient such as example modalities 105 and 110, or brachytherapy, where the source 125 is placed within the patient such as example modality 115. Therefore, a first modality may be a photon beam with an energy level of 6 MV and a second modality may be a photon beam with an energy level of 15 MV. Similarly, a first modality may be a proton beam with a 200 MeV energy level and a second modality may be a photon beam with a 6 MV energy level. Other variants are considered.

The radiation modalities may be delivered in the same treatment room with the same device in different modes of operation, or with different devices altogether. In some embodiments, an x-ray photon beam and an electron beam may be treated with the same particle accelerator. The photons are delivered by converting high energy electrons into photons via a bremsstrahlung target, and in some cases flattening the beam with a flattening filter. Electrons are delivered without the presence of the target, and the beam may in some cases be flattened with a flattening filter. An electron applicator may also be affixed to the gantry or a multileaf collimator (MLC) may be used for better beam collimation. In other embodiments, an x-ray photon modality and a proton modality are treated separately, in different rooms, with different delivery devices. In other embodiments, external beam treatments delivered with a linear accelerator are combined with brachytherapy treatments delivered with a remote afterloader device.

The radiation modalities have various degrees of freedom which may be varied throughout the patient treatment in order to deliver a dose distribution, such as gantry angle, MLC leaf positions, fluence maps, beam energy, collimator angle, and patient support collimator angle. These may be subdivided into delivery elements. For external beam radiotherapy, delivery elements may be a set of delivery elements formed by multi-leaf collimators from fixed or rotating gantry angles, or a set of fluence maps that can subsequently be converted to deliverable multi-leaf collimator movements. For brachytherapy, delivery elements may be a series of source positions of a radioactive source along a catheter within a delivery applicator. Each delivery element has an associated weight, which may correspond to an absolute or relative dose weighting, or a length of time over which each delivery element may be delivered. Therefore, a modality may differ in its delivery elements but still be considered a same modality.

Prior to treating a subject with one or more modalities, one or more images 170 may be acquired and stored. Example image acquisition techniques may be, but are not limited to, computerized tomography (CT), magnetic resonance imaging (MRI), position emission tomography (PET) and ultrasound. Various representations of the subject's anatomy may be identified on the one or more images, such as targets and organs at risk, which may also be stored as structure sets with the images 170.

Generally, a set of one or more goals regarding a target dose distribution are defined in order to design a treatment plan based on the images 170 or derived structure sets. The goals may be defined, for example, as tolerances and dose coverage constraints to targets and organs at risk identified in the images, for example that 90% of the target must be above a prescribed dose, such as 60 Gy and that 50% of organs at risk must receive less than a tolerance dose, such as 50 Gy. Other goals are also applicable.

A treatment plan consists of delivery elements, such as apertures, and corresponding weights. Dose distributions can be calculated on the images 170 with a dose engine 150. The dose engine 150 may use measured data, Monte Carlo algorithms, superposition/convolution algorithms, collapsed cone algorithms, or any other type of algorithm that computes radiation doses on medical images. The dose engine 150 may include tissue inhomogeneity effects using, for example, calibrated pixel values from the images 170.

An optimizer 155 finds a set of delivery elements and associated weights that generate a simulated dose distribution on the images 170 that satisfy, as closely as possible, the goals. A cost function, or metric, is designed which is low when the goals are fulfilled, and high when they are not (or, in some cases, vice-versa). The optimizer 155 may find the set of delivery elements which minimizes the cost function, or a set of optimal weights given a fixed set of delivery elements.

The cost function may in some embodiments be defined in terms of dose-volume constraints, voxel-based penalty functions, tumor control probability (TCP) metrics, equivalent uniform dose (EUD) metrics, mean dose to organs, conditional value at risk (CVaR), and the like.

In some embodiments, the cost function is assumed to be a convex function. In others, but not in all, a non-convex function is represented as a convex function through approximation of local characteristics.

In some embodiments, delivery elements are uniquely defined radiation-emitting elements, such as a source position for brachytherapy treatments, or a beamlet of fluence across a photon, electron or proton field. In other embodiments, delivery elements are combinations of uniquely defined radiation-emitting elements, such as multi-leaf collimator apertures from unique gantry angles, which may contain a finite number of beamlets within the aperture.

Optimization of the cost function may be performed iteratively. An initial set of delivery elements may first be defined. In some embodiments, the initial set is a null set, containing no delivery elements. At each iteration, a single delivery element, selected from the modalities may be added to the set of delivery elements, although in some embodiments multiple delivery elements may be added in a single iteration. The selected modality is chosen by determining which of the modalities has the highest potential to step closer towards reaching the optimization goals. After adding one or more new delivery elements to the set from the selected modality, the weights of the new set of delivery elements are adjusted in an attempt to move a step closer towards optimizing the cost function. In some iterations, delivery elements may be removed if they no longer contribute strongly towards reaching the optimal solution.

In some embodiments, at each iteration, the modality is selected through calculation of a decision variable for each potential delivery element k, where the potential delivery elements considered belong to the entire set of delivery elements from all modalities combined. The decision variable may be based on an approximation of the cost function, such as a linearization or a Taylor expansion about the current point in solution space.

In some implementations, doses from different delivery elements from different modalities will be normalized, e.g., by a maximum dose pertaining to a group of delivery elements, for example for every beamlet in a photon fluence map, or every deliverable electron aperture. This may help ensure an adequate representation from each delivery element from disparate modalities.

In some embodiments, after addition of a delivery element from a selected modality, conditions for optimality may be verified to ensure that adding the delivery element successfully helps steer the solution towards optimizing the cost function. As an example, combining Karush-Kuhn-Tucker (KTT) conditions can be reduced to an example condition

$${\sum }_{j}^{ } {D}_{k , j} {π}_{j} \geq 0$$

where Dₖⱼ is the dose to voxel j from delivery element k,

$${π}_{j} = \frac{\partial F}{\partial {z}_{j}}$$

where F(z) is the cost function and zⱼ is the total dose in voxel j. This may serve as an optimality test and may be used, in some cases, as a method to create potential delivery elements. The πⱼ may be obtained after optimizing the current set of aperture weights, and the Dₖⱼ are the unit dose deposition coefficients for any valid delivery element, including those not currently in the set of delivery elements. If all possible delivery elements satisfy the example condition equation, then the set of delivery elements in the current iteration is optimal in the sense that the cost function cannot be improved by adding more delivery elements. On the other hand, any delivery element violating the example condition equation is a potential candidate for addition to the current set of multimodality delivery elements. The goal may then be to find delivery elements which violate the example condition. Strategies to finding potential delivery elements may also include limiting to allowable delivery elements, such as apertures which may be delivered by a multileaf collimator, or source positions that may be reached with a catheter. In some implementations, in order to create the highest quality multi-modality treatment plan with the fewest delivery elements, the delivery element which violates the example condition the most may be added to the set of delivery elements in a given iteration.

Once the optimal solution is reached, delivery elements and weights may be converted into machine readable instructions for controlling the two or more modalities. As an example, apertures and beam angles may be generated for a photon treatment plan and an electron treatment plan. The instructions may be sent electronically to a record and verify system, which stores the information for subsequent treatments, and is configured to control the modalities during treatment delivery. The subject may, for example, be set up for treatment once per day for a duration of 5 weeks. Each day, for example, a photon plan may first be delivered, immediately followed by an electron plan. The total dose delivered to the subject will then have been delivered in an optimally combined manner.

The processor 145 may be implemented, for example, using one or more central processing units (CPUs), with each CPU having one or more processing cores. The processor 145 may perform tasks using software (e.g., executable instructions) stored in the memory 165, for example. Additionally, the processor 145 may calculate dose distributions, delivery elements and weights and cause them to be stored. Processing tasks may also be implemented, in some embodiments using one or more graphical processing units (GPUs).

The memory 165 may be generally any electronic storage, including volatile or nonvolatile memory, which may encode instructions for performing functions described herein.

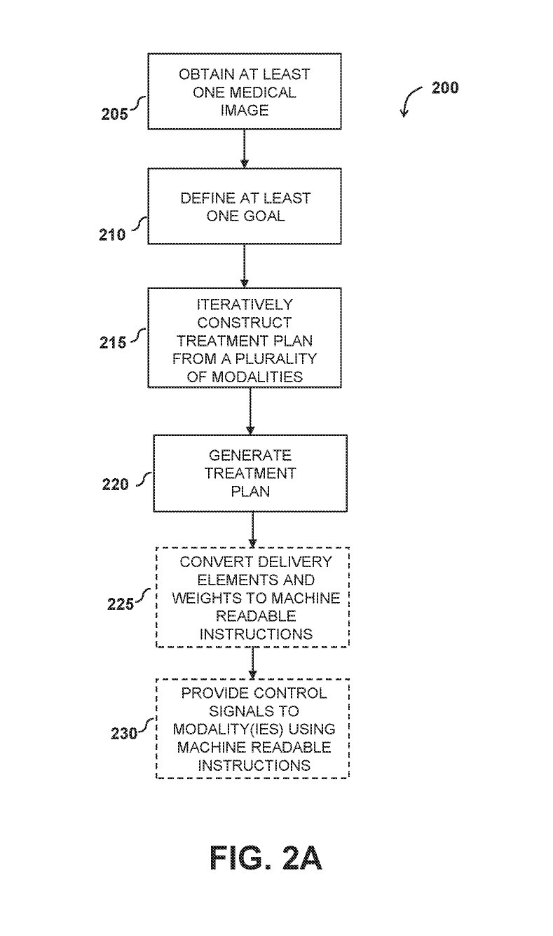

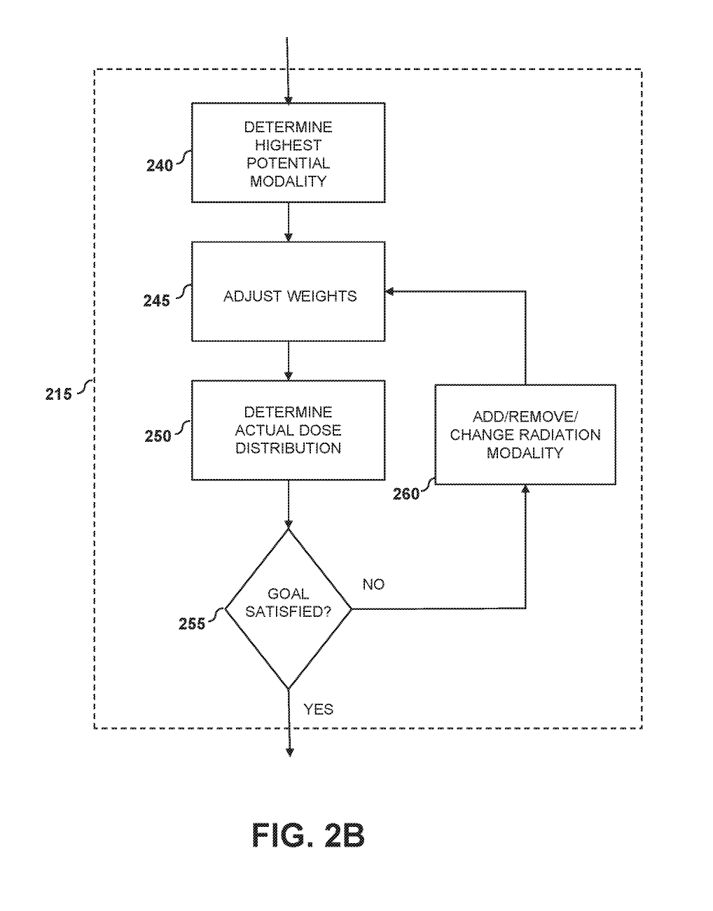

FIG. 2A is an example method 200 for generating a treatment plan in accordance with at least some embodiments of the present disclosure. The operations described in the blocks 205 through 230 may be performed in response to execution (such as by one or more processors described herein) of computer-executable instructions stored in a computer-readable medium, such as a computer-readable medium of a computing device or some other controller similarly configured.

An example process may begin with block 205, where at least one medical image of the subject is obtained. In some embodiments, obtaining the medical image comprises acquiring the medical image using an image acquisition device, such as a CT, PET, US, and MRI device. In some embodiments, obtaining the medical image comprises retrieving stored images from a local or remote storage medium. In some embodiments, a CT image of a patient's complete body is obtained using a CT simulator. In some embodiments, an MRI image, PET image and/or an ultrasound image may be acquired and registered to the CT image.

Block 205 may be followed by block 210, where at least one goal is defined. The goal is defined with regards to a target dose distribution to at least a portion of at least one of the images. This may be, for example, a quantity to be extracted from a dose distribution. In some embodiments, targets and organs at risk may first be outlined on the one or more medical images and stored as treatment planning structures. The images and planning structures may be analyzed and dose tolerances and/or or biological tolerances may be defined on targets and organs at risk. These may be defined, for example, as maximum volume percentages of a structure which may reach a defined level of dose, or a maximum dose to a defined percentage volume of a structure. Multiple goals may be defined per planning structure. In some embodiments, the goals are defined using an automated tool, such as a neural network or other form of artificial intelligence capable of applying dose tolerances and/or biological tolerances on targets and/or organs at risk.

Block 210 may be followed by block 215, where a treatment plan is iteratively constructed from a plurality of modalities. At least one radiation modality is selected from the plurality of radiation modalities for the treatment plan. In some embodiments, the treatment plan comprises at least two radiation modalities. Each radiation modality has at least one delivery element from a plurality of delivery elements and at least one associated weight, which may be, for example, an absolute or relative dose, or a time increment.

In some embodiments, an initial set of delivery elements used for the iterative construction may be derived from an initial approximate treatment plan, or a treatment plan that has been delivered in a previous treatment given to the same patient, for example on a previous day or during a previous series of treatments. The treatment plan is iteratively constructed until a condition associated with the goal(s) is met. Block 215 will be explained in greater detail with reference to FIG. 2B.

Block 215 may be followed by block 220, where the treatment plan is generated based on the at least one delivery element and at least one associated weight, for delivery of the radiation dose by the at least one radiation modality.

In some embodiments, method 200 comprises block 225, where the delivery elements and associated weights are converted to machine readable instructions. These may be stored for future use and/or provided to the one or more modalities as a set of control signals, as per block 230. The method 200 may thus comprise steps of controlling the radiation modalities for delivery of the radiation dose(s) to the subject in accordance with the treatment plan.

Referring to FIG. 2B, there is illustrated an example embodiment of block 215. At block 240 a highest potential modality is determined. The highest potential modality corresponds to the modality from the plurality of modalities having the greatest likelihood of reaching the goal(s). Each modality from the plurality of modalities may itself be associated with a highest potential delivery element and associated weight. In some embodiments, block 240 is performed in two steps. In a first step, each modality is optimized to be associated with a highest potential delivery element for that modality. In a second step, the highest potential modality is selected from the modalities associated with the highest potential delivery elements.

Determining the highest potential modality at block 240 may involve approximating a cost function based around a current point in solution space, for example using a Taylor series expansion, by finding delivery elements that violate conditions such as Karush-Kuhn Tucker conditions, by finding delivery items that violate conditions the most, or any other similar method.

Block 240 may be followed by block 245, where the weights for the delivery elements of the highest potential modality are adjusted so as to move towards the goal(s). Weights for the current set of delivery elements in a given iteration may be determined at block 245 by optimizing a cost function constructed using the goal with an optimization engine.

Block 245 may be followed by block 250, where an actual dose distribution is determined based on the image(s), using the delivery element(s) and associated weight(s) of the highest potential modality.

At decision block 255, an evaluation is made as to whether the condition (or termination criteria) associated with the goal is met by the actual dose distribution. If the condition has not been reached, the method 200 returns to block 245 and repeats blocks 245, 250 and 255. If the condition has been reached, the method 200 moves on to block 220 (of FIG. 2A). In some example embodiments, iteration is complete when the goals are satisfied. Alternatively or in combination therewith, iteration is complete when a cost function cannot be improved by more than a threshold through adding more delivery elements and/or modalities.

To convert delivery elements and weights to machine readable instructions, as per block 225, in some embodiments a verification is made that all delivery elements are actually deliverable with the physical treatment modalities, although this may have already been added as a constraint during the iterative construction of the treatment plan. Delivery elements are converted to, for example, MLC shapes, movements, dose rates, gantry angles, collimator angles, patient support apparatus angles, brachytherapy source positions, and the like, and sent to, for example, a record and verify system for delivery with each modality over a series of treatment sessions.

If machine readable instructions are sent to a record and verify system, for example, patients are then treated with the information contained therein, as per block 230. In some cases, the same treatment device is used to deliver multiple modality treatments in sequence. For example, photon and electron treatments can be delivered with conventional linear accelerators under different modes of operation. In other cases, completely different devices are used, for example if proton and brachytherapy modalities are combined.

The blocks included FIGS. 2A and 2B are for illustration purposes. In some embodiments, the blocks may be performed in a different order. In some other embodiments, various blocks may be eliminated. In still other embodiments, various blocks may be divided into additional blocks, supplemented with other blocks, or combined together into fewer blocks. Other variations of these specific blocks are contemplated, including changes in the order of the blocks, changes in the content of the blocks being split or combined into other blocks, and the like.

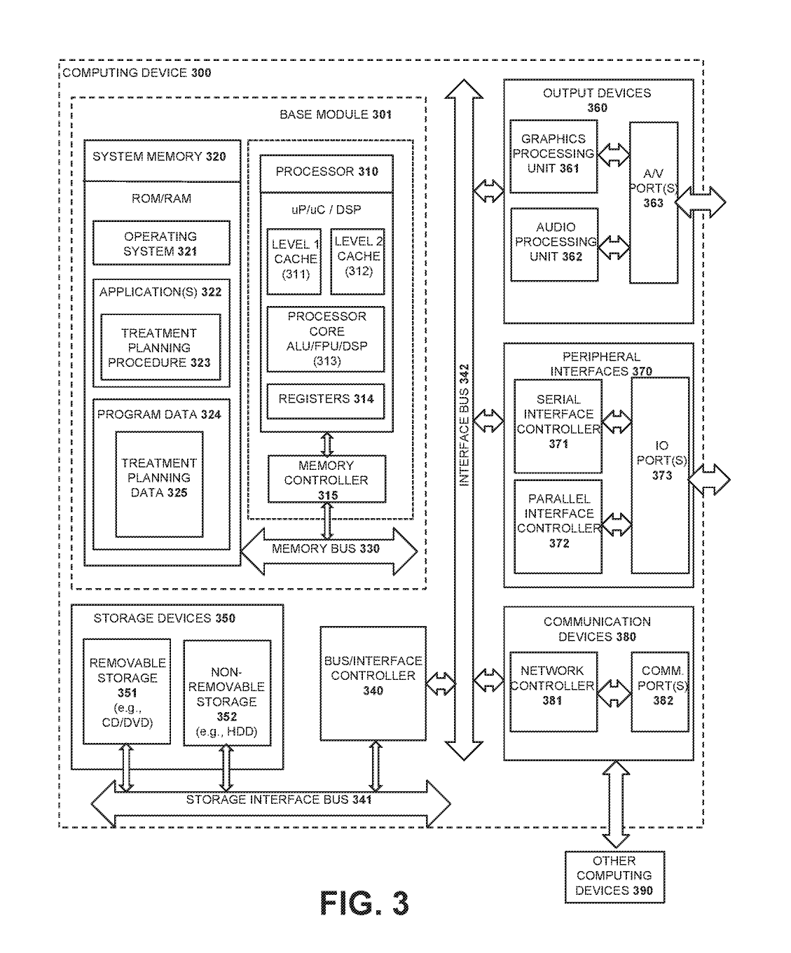

FIG. 3 is a block diagram illustrating an example embodiment of a computing device 300 that is arranged for providing modality treatments in accordance with the present disclosure. In some embodiments, computing device 300 includes one or more processors 310 and system memory 320 comprised in a base module 301. A memory bus 330 may be used for communicating between the processor 310 and the system memory 320.

Depending on the desired configuration, processor 310 may be of any type including but not limited to a microprocessor (μP), a microcontroller (μC), a digital signal processor (DSP), or any combination thereof. Processor 310 may include one more levels of caching, such as a level one cache 311 and a level two cache 312, a processor core 313, and registers 314. An example processor core 313 may include an arithmetic logic unit (ALU), a floating point unit (FPU), a digital signal processing core (DSP Core), or any combination thereof. An example memory controller 315 may also be used with the processor 310, or in some implementations the memory controller 315 may be an internal part of the processor 310.

Depending on the desired configuration, the system memory 320 may be of any type including but not limited to volatile memory (such as RAM), non-volatile memory (such as ROM, flash memory, etc.) or any combination thereof. System memory 320 may include an operating system 321, one or more applications 322, and program data 324. Application(s) 322 may include a treatment planning procedure 323 that is arranged to provide multimodality treatment plan as described herein. Program data 324 may include treatment planning data 325, which may comprise one or more medical images, delivery elements, weights, goals, and/or other information useful for the generation and implementation of the treatment plan. In some embodiments, application(s) 322 may be arranged to operate with program data 324 on an operating system 321 such that any of the procedures described herein may be performed. This described configuration is illustrated in FIG. 3 by those components within the base module 301.

Computing device 300 may have additional features or functionality, and additional interfaces to facilitate communications between the base module 301 and any other devices and interfaces. For example, a bus/interface controller 340 may be used to facilitate communications between the base module 301 and one or more storage devices 350 via a storage interface bus 341. The storage devices 350 may be removable storage devices 351, non-removable storage devices 352, or a combination thereof. Examples of removable storage and non-removable storage devices comprise magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

System memory 320, removable storage 351 and non-removable storage 352 are all examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 300. Any such computer storage media may be part of computing device 300.

Computing device 300 may also include an interface bus 342 for facilitating communication from various interface devices (e.g., output interfaces, peripheral interfaces, and communication interfaces) to the base module 301 via the bus/interface controller 340. Example output devices 360 include a graphics processing unit 361 and an audio processing unit 362, which may be configured to communicate to various external devices such as a display or speakers via one or more A/V ports 363. Example peripheral interfaces 370 comprise a serial interface controller 371 or a parallel interface controller 372, which may be configured to communicate with external devices such as input devices (e.g., keyboard, mouse, pen, voice input device, touch input device, etc.) or other peripheral devices (e.g., printer, scanner, etc.) via one or more I/O ports 373. An example communication device 380 comprises a network controller 381, which may be arranged to facilitate communications with one or more other computing devices 390 over a network communication link via one or more communication ports 382.

The network communication link may be one example of a communication media. Communication media may be embodied by computer readable instructions, data structures, program modules, or other data in a modulated data signal, such as a carrier wave or other transport mechanism, and may include any information delivery media. A modulated data signal may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

Computing device 300 may be implemented as a portion of a small-form factor portable (or mobile) electronic device such as a cell phone, a personal data assistant (PDA), a personal media player device, a wireless web-watch device, a personal headset device, an application specific device, or a hybrid device that includes any of the above functions. Computing device 300 may also be implemented as a personal computer including both laptop computer and non-laptop computer configurations.

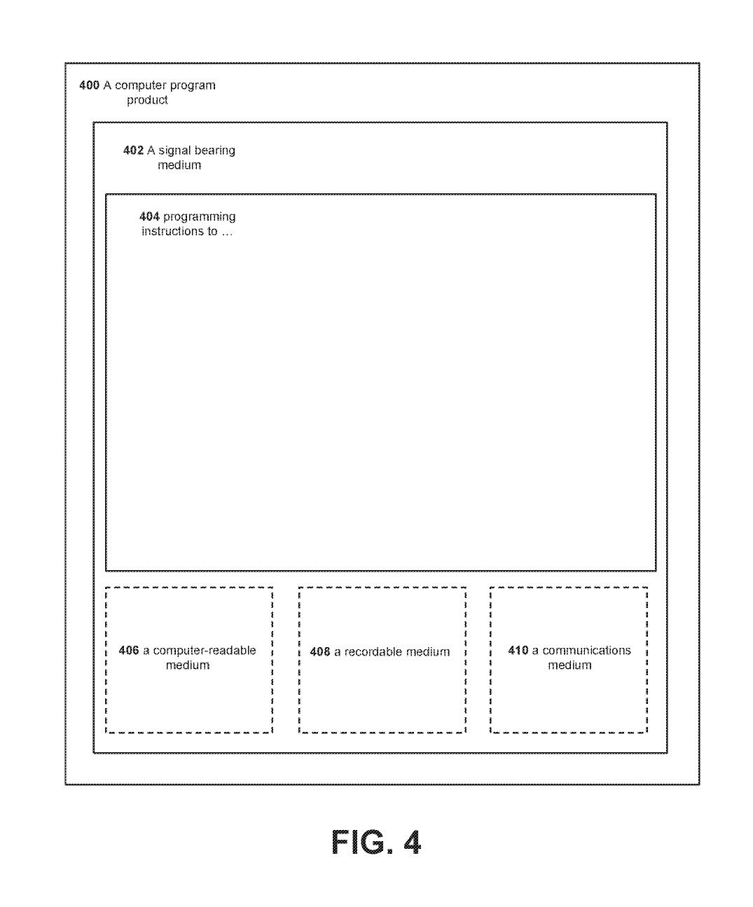

FIG. 4 is a block diagram illustrating an example computer program product 400 that is arranged to store instructions for delivering treatments in accordance with the present disclosure. The signal bearing medium 402 which may be implemented as or include a computer-readable medium 406, a computer recordable medium 408, a computer communications medium 410, or combinations thereof, stores programming instructions 404 that may configure the processing unit to perform all or some of the processes previously described. These instructions may include, for example, one or more executable instructions for causing a processor to obtain at least one medical image of the subject; define at least one goal regarding a target dose distribution to at least a portion of the at least one image; iteratively construct the treatment plan by selecting at least one radiation modality from a plurality of radiation modalities, the at least one radiation modality having at least one delivery element from a plurality of delivery elements and at least one associated weight, until a condition associated with the at least one goal is met; and generate the treatment plan based on the at least one delivery element and at least one associated weight, for delivery of the radiation dose by the at least one radiation modality.

The present disclosure is not to be limited in terms of the particular examples described herein, which are intended as illustrations of various aspects. Many modifications and examples can be made, as will be apparent to those skilled in the art. Functionally equivalent methods and apparatuses within the scope of the disclosure, in addition to those enumerated herein, will be apparent to those skilled in the art from the foregoing descriptions. Such modifications and examples are intended to fall within the scope of the appended claims. The present disclosure is to be limited only by the terms of the appended claims, along with the full scope of equivalents to which such claims are entitled.

It will be understood by those within the art that, in general, terms used herein, and especially in the appended claims (e.g., bodies of the appended claims) are intended as “open” terms (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to examples containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 items refers to groups having 1, 2, or 3 items. Similarly, a group having 1-5 items refers to groups having 1, 2, 3, 4, or 5 items, and so forth.

While the foregoing detailed description has set forth various examples of the devices and/or processes via the use of block diagrams, flowcharts, and/or examples, such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one example, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the examples disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. For example, if a user determines that speed and accuracy are paramount, the user may opt for a mainly hardware and/or firmware vehicle; if flexibility is paramount, the user may opt for a mainly software implementation; or, yet again alternatively, the user may opt for some combination of hardware, software, and/or firmware.

In addition, those skilled in the art will appreciate that the mechanisms of the subject matter described herein are capable of being distributed as a program product in a variety of forms, and that an illustrative example of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

While various aspects and examples have been disclosed herein, other aspects and examples will be apparent to those skilled in the art. The various aspects and examples disclosed herein are for purposes of illustration and are not intended to be limiting, with the true scope and spirit being indicated by the following claims.

1. A method for generating a treatment plan for delivery of a radiation dose to a subject, the method comprising:
obtaining at least one medical image of the subject;
defining at least one goal regarding at least a portion of the at least one image;
iteratively constructing the treatment plan by selecting at least one delivery element from a plurality of delivery elements, the at least one delivery element having at least one associated weight, until a condition associated with the at least one goal is met and a cost function is optimized; and
generating the treatment plan based on the at least one delivery element and the at least one associated weight, for delivery of the radiation dose by at least one radiation modality.

2. The method of claim 1, wherein iteratively constructing the treatment plan comprises combining at least two modalities from a plurality of modalities to satisfy the at least one goal.

3. The method of claim 1, wherein iteratively constructing the treatment plan comprises:
(a) determining a highest potential radiation modality from a plurality of radiation modalities, the highest potential modality having a greatest likelihood of reaching the at least one goal;
(b) adjusting the at least one weight associated with the at least one delivery element to move towards the at least one goal;
(c) determining an actual dose distribution on the at least one image using the at least one weight and the at least one delivery element; and
(d) adding, removing, or changing a radiation modality and repeating (b) and (c) until the condition associated with the at least one goal is met by the actual dose distribution.

4.-15. (canceled)

16. The method of claim 1, wherein the at least one associated weight comprises a scaling factor of relative or absolute doses for a corresponding delivery element.

17. The method of claim 1, wherein the at least one associated weight comprises a relative or absolute treatment time for a corresponding delivery element.

18. The method of claim 3, wherein a highest potential radiation modality comprises computing Karush-Kuhn Tucker conditions.

19. The method of claim 3, wherein a highest potential radiation modality comprises approximating the cost function about a current point in a solution space.

20. The method of claim 19, wherein approximating the cost function comprises using a Taylor expansion.

21. The method of claim 3, wherein adding, removing, or changing a radiation modality comprises finding delivery elements that violate a condition.

22. The method of claim 21, wherein adding, removing, or changing a radiation modality comprises finding delivery elements that violate a condition by a highest amount.

23.-28. (canceled)

29. A system for generating a treatment plan for delivery of a radiation dose to a subject, the system comprising:
at least one processor; and
a non-transitory computer-readable medium having stored thereon program code executable by the at least one processor for:
obtaining at least one medical image of the subject;
receiving at least one goal regarding at least a portion of the at least one image;
iteratively constructing the treatment plan by selecting at least one delivery element from a plurality of delivery elements, the at least one delivery element having at least one associated weight, until a condition associated with the at least one goal is met and a cost function is optimized; and
generating the treatment plan based on the at least one delivery element and the at least one associated weight, for delivery of the radiation dose by at least one radiation modality.

30. The system of claim 29, wherein iteratively constructing the treatment plan comprises combining at least two modalities from a plurality of modalities to satisfy the at least one goal.

31. The system of claim 29, wherein iteratively constructing the treatment plan comprises:
(a) determining a highest potential radiation modality from a plurality of radiation modalities, the highest potential modality having a greatest likelihood of reaching the at least one goal;
(b) adjusting the at least one weight associated with the at least one delivery element to move towards the at least one goal;
(c) determining an actual dose distribution on the at least one image using the at least one weight and the at least one delivery element; and
(d) adding, removing, or changing a radiation modality and repeating (b) and (c) until the condition associated with the at least one goal is met by the actual dose distribution.

32. The system of claim 29, wherein the at least one associated weight comprises a scaling factor of relative or absolute doses for a corresponding delivery element.

33. The system of claim 31, wherein the at least one associated weight comprises a relative or absolute treatment time for a corresponding delivery element.

34. The system of claim 31, wherein a highest potential radiation modality comprises computing Karush-Kuhn Tucker conditions.

35. The system of claim 31, wherein a highest potential radiation modality comprises approximating the cost function about a current point in a solution space.

36. The system of claim 35, wherein approximating the cost function comprises using a Taylor expansion.

37. The system of claim 31, wherein adding, removing, or changing a radiation modality comprises finding delivery elements that violate a condition.

38. The system of claim 37, wherein adding, removing, or changing a radiation modality comprises finding delivery elements that violate a condition by a highest amount.
